(19) United States
(12) Patent Application Publication
(10) Pub. No.: US 2004/0009196 A1
(43) Pub. Date: Jan. 15, 2004

(54) Use of dhea and some of its derivatives in a cosmetic composition for preventing or treating dermal atrophy

(76) Inventors: Corrine Ferraris, Paris (FR); Isabelle Castiel, Jouy en Josas (FR); Olivia Zobiri, Clichy (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/275,286

(22) PCT Filed: May 3, 2001
(86) PCT No.: PCT/FR01/01352

(30) Foreign Application Priority Data
May 15, 2000 (FR) 0006154

(51) Int. Cl. A61K 31/57; A61K 7/00

(52) U.S. Cl. 424/401; 514/177

(57) ABSTRACT

The present invention relates to the use of DHEA and/or of its chemical and/or biological precursors and derivatives, in a cosmetic composition or for the manufacture of a dermatological composition for topical application to the skin, for preventing atrophy of the epidermis which is one of the characteristic signs of intrinsic and/or photoinduced skin ageing.

The invention relates to the use of DHEA and/or its chemical and/or biological precursors and derivatives, in a cosmetic composition or for the manufacture of a dermatological composition for topical application to the skin, for preventing atrophy of the epidermis.

Human skin consists of two compartments, namely a deep compartment, the dermis, and a top compartment, the epidermis.

The dermis provides the epidermis with a solid support. It is also its feeder component. It is mainly composed of fibroblasts and an extracellular matrix which is itself mainly composed of collagen, elastin and a substance, called ground substance; which components are synthesized by the fibroblast. Leukocytes, mastocytes or tissue macrophages are also present therein. It also contains blood vessels and nerve fibres.

The epidermis is in contact with the external environment. Its role consists in protecting the body from dehydration and from external attacks, whether they are chemical, mechanical, physical or infectious.

The natural human epidermis is mainly composed of three types of cell which are the keratinocytes, which are highly predominant, the melanocytes and the Langerhans cells. Each of these cell types contributes, by its specific functions, to the essential role played in the body by the skin.

The cells constituting the epidermis are delimited by a lipid domain. The epidermal lipids are mainly synthesized in the living epidermis. They are essentially composed of phospholipids, sphingolipids, cholesterol, free fatty acids, triglycerides, esters of cholesterol and alkanes. During cell differentiation, the phospholipids, whose role consists in producing the fluid structure of the cell membranes of the living layers of the epidermis, are gradually replaced by a mixture which is predominantly composed of fatty acids, cholesterol and sphingolipids, which are essential constituents of the horny layer of the epidermis (stratum corneum).

The lipids of the intercorneocyte cement of the skin, and in particular the ceramides, are organized into lamellar bilayers or sheets and participate in the cohesion of the stratum corneum in order to maintain the integrity of the barrier and its protective, antipenetration and anti-irritation role, and the like.

It can be understood why activation of the metabolism in the living cells of the epidermis, or an increase in cell proliferation in the living layers, will result in an increase in the epidermal content of phospholipids (sphingomyelin/phosphatidylinositol or phospholipids of the membranes, respectively) and will result in an increase in the size or in the number of living cells, that is to say in a thickening of the epidermis.

However, it is known that during chronobiological ageing, in particular at the menopause, atrophy of the epidermis is observed which results from a general slowing of cellular metabolism and which is partly responsible for the appearance of wrinkles and fine lines. Atrophy of the epidermis has also been identified as one of the histological signs of photoageing (Gilchrest B. A., Skin and Aging Processes, 1989, CRC Press).

It can therefore be understood why it is important to have a means for facilitating cell multiplication or metabolism, in particular of the living cells of the epidermis, for combating atrophy of the epidermis and thus giving the skin a young appearance again.

There has already been described in U.S. Pat. No. 5,843,932 the use of DHEA for remedying the atrophy of the dermis by inhibiting the loss of collagen and of connective tissue. There has additionally been described in U.S. Pat. No. 5,736,537 the oral use of DHEA esters, in particular of DHEA salicylate, for regulating skin atrophy caused by thinning or general degradation of the dermis.

DHEA, or dehydroepiandrosterone, is a natural steroid essentially produced by the adrenocortical glands. The exogenous DHEA, administered by the topical or oral route, is also known for its capacity to promote keratinization of the epidermis (JP-07 196 467) and to treat dry skins by increasing the endogenous production and the secretion of sebum and by thereby reinforcing the barrier effect of the skin (U.S. Pat. No. 4,496,556).

However, to the knowledge of the Applicant, it has never yet been suggested that DHEA applied by the topical route could have any effect on the atrophy of the epidermis. In particular, the anti-atrophying effect of DHEA on the dermis did not suggest that this compound could have an effect on the epidermis, the cells involved and the mechanisms of action being quite distinct in these two compartments of the skin.

The subject of the present invention is therefore the use of DHEA and/or of its chemical and/or biological precursors and derivatives, in a cosmetic composition for topical application to the skin, as agent for preventing atrophy of the epidermis.

The invention also relates to the use of DHEA and/or of its chemical and/or biological precursors and derivatives for the manufacture of a composition for topical application to the skin, intended to prevent atrophy of the epidermis.

The invention finally relates to the use of DHEA and/or of its chemical and/or biological precursors and derivatives, in a cosmetic composition for topical application to the skin, as agent for preventing the signs of skin ageing linked to atrophy of the epidermis.

DHEA has the following formula (I):

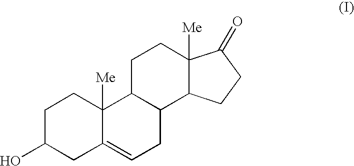

The DHEA which can be used according to the invention is for example available from the company AKZO NOBEL.

The expression DHEA precursors is understood to mean its immediate biological precursors or substrates as well as its chemical precursors. Examples of biological precursors are Δ⁵-pregnenolone, 17α-hydroxypregnenolone and 17α-hydroxypregnenolone sulphate, without this list being limiting. Examples of chemical precursors are sapogenins such as diosgenin (or spirost-5-en-3-beta-ol), hecogenin, hecogenin acetate, smilagenin and sarsapogenin, and natural extracts containing them, in particular fenugreek and extracts of Dioscorea such as the root of wild yam, without this list being limiting.

The expression DHEA derivatives is understood to mean both its biological derivatives and its chemical derivatives. As biological derivatives, there may be mentioned in particular Δ⁵-androstene-3,17-diol and Δ⁴-androstene-3,17-dione, without this list being limiting. As chemical derivatives, there may be mentioned in particular salts, in particular the water-soluble salts, such as DHEA sulphate. There may also be mentioned esters, such as esters of hydroxycarboxylic acids and of DHEA described in U.S. Pat. No. 5,736,537 or the other esters such as DHEA salicylate, acetate, valerate and enanthate.

The composition containing DHEA and/or at least one of its precursors or derivatives is appropriate for topical use and it therefore contains a physiologically acceptable medium, that is to say which is compatible with the skin.

This composition may contain from 10⁻⁶ to 10% by weight, advantageously from 0.1 to 5% by weight, and even better about 1% by weight of DHEA and/or its precursor or derivative, relative to the total weight of the composition.

It may be provided in all the galenic forms normally used in the cosmetic and dermatological fields and it may be in particular in the form of an optionally gelled aqueous solution, of an optionally two-phase lotion-type dispersion, of an emulsion obtained by dispersing a fatty phase in an aqueous phase (O/W) or conversely (W/O), or of a triple emulsion (W/O/W or O/W/O) or of an ionic and/or non-ionic type vesicular dispersion. These compositions are prepared according to the customary methods.

This composition may be more or less fluid and may have the appearance of a white or coloured cream, of an ointment, of a milk, of a lotion, of a serum, of a paste or of a foam. It may be optionally applied to the skin in the form of an aerosol. It may also be provided in the form of a solid, in particular in the form of a stick. It may be used as a care product and/or a make-up product for the skin.

In a known manner, the composition of the invention may also contain the usual adjuvants in the cosmetic field, such as hydrophilic or lipophilic gelling agents, hydrophilic or lipophilic active agents, preservatives, antioxidants, solvents, perfumes, fillers, screening agents, pigments, odour absorbers and colouring matter. The quantities of these various adjuvants are those conventionally used in the field considered, and for example from 0.01 to 20% of the total weight of the composition. These adjuvants, depending on their nature, may be introduced into the fatty phase, into the aqueous phase, into lipid vesicles and/or into nanoparticles. In any case, these adjuvants, and their proportions, will be chosen so as not to adversely affect the desire properties of the combination of active agents according to the invention.

When the composition of the invention is an emulsion, the proportion of the fatty phase may range from 5 to 80% by weight, and preferably from 5 to 50% by weight relative to the total weight of the composition. The oils, emulsifiers an coemulsifiers used in the composition in the form of an emulsion are chosen from those conventionally used in the field considered. The emulsifier and coemulsifier are present in the composition in a proportion ranging from 0.5 to 20% by weight relative to the total weight of the compostion.

As oils which may be used in the invention, there may be mentioned mineral oils (liquid paraffin), oils of plant origin (avocado oil, soybean oil), oils of animal origin (lanolin), synthenic oils (perhydrosqualene), silicone oils (cyclomethicone) and fluorinated oils(perflouropolyethers). It is also possible to use, as fatty substances, fatty alcohols (cetyl alcohol), fatty acids, waxes (carnauba wax ozokerite).

As emulsifiers and coemulsifiers which can be used in the invention, there may be mentioned, for example, esters of fatty acid and of polyethylene glycol such as PEG-20 stearate, and esters of fatty acid and of glycerine such as glyceryl stearate.

As hydrophilic gelling agents, there may be mentioned in particular carboxyvinyl polymers (carbomer), acrylic copolymers such as copolymers of acrylates/alkyl acrylates, polyacrylamides, polysaccharides, natural gums and clays, and, as lipophilic gelling agents, there may be mentioned modified clays such as bentones, metal salts of fatty acids, hydrophobic silica and polyethylenes.

The invention will be understood more clearly, and its advantages will emerge more clearly, in the light of the following examples, which are given by way of illustration, and without limitation.

### EXAMPLES

### Example 1

### Effect of DHEA Applied by the Topical Route in Vitro

In this example, 2 μl of DHEA at 1×10⁻⁴ M in ethanol were applied every 48 hours, for 6 days, to a skin equivalent sold by the company EPISKIN (LYON, France), after culturing it for 7 days and incubating overnight with [¹⁴C]acetate (2 μCi/ml). The culture and test media are those contained in the kit sold by the supplier. The control consists of an identical epidermal equivalent undergoing a topical application of 2 μl of ethanol diluted 1/1 000.

At the end of the incubation, the epidermal equivalent is detached from its collagenic support. The preparation of the lipids of the epidermal equivalent, and their analysis by HPTLC or high performance thin-layer chromatography, are carried out according to the technique and with the buffers described by M. Ponec (1991, Adv. Lipid Res., 24: 83-117). At the end of the migration of the lipids, autoradiography of the chromatography plate is carried out overnight.

An increase of 35% in the epidermal content of phospholipids is observed compared with the control.

It is therefore clearly evident that the topical application of DHEA to the reconstructed epidermis induces a significant increase in the epidermal content of phospholipids.

In parallel, no modification in epidermal differentiation is observed, as measured by the expression of transglutaminase and of filaggrin (immunofluorescence labelling) which are two proteins used as terminal differentiation markers, transglutaminase being the key enzyme responsible for the formation of the horny envelope (Rice et al., Relation of protein synthesis and transglutaminase activity to formation of the cross-linked envelope during terminal differentiation of the cultured human epidermal keratinocyte, J. Cell Biol. 1978, 76: p. 705-711). This is confirmed by the histological analysis of the samples, which shows no increase in the number of granular layers or in parakeratosis.

It follows that the increase noted above in the epidermal content of phospholipids is not correlated with an increase in keratinization.

### Example 2

### Cosmetic Composition

| | |
|---|---|
| Phase A1 | |
| 2-Octyldodecanol | 20% |
| DHEA | 1% |
| Phase A2 | |
| Polyglyceryl distearate (2 mol) | 2% |
| PEG monostearate (8 EO) | 1.35% |
| Stearic acid | 1% |
| Preservative | 0.1% |
| Phase B | |
| Preservatives | 0.35% |
| Neutralizing agents | 0.25% |
| Propylene glycol | 10% |
| Water | qs 100 |
| Phase C | |
| Gelling agent | 0.5% |
| Neutralizing agent | 0.2% |
| Water | qs |

This composition was prepared in the following manner: phases A1, A2 and B were prepared separately by mixing their constituents in the hot state, with stirring. Phases A1 and A2 were mixed in the hot state, and then phase B was added thereto. The mixture thus obtained was transferred to a high-pressure homogenizer where it was subjected to three passes at 600 bar before incorporating phase C.

This composition may be used as twice daily applications for preventing atrophy of the epidermis, in particular in menopausal women.

1. Use of DHEA and/or of its chemical and/or biological precursors and derivatives, in a cosmetic composition for topical application to the skin, as agent for preventing atrophy of the epidermis.

2. Use of DHEA and/or of its chemical and/or biological precursors and derivatives for the manufacture of a composition for topical application to the skin, intended to prevent atrophy of the epidermis.

3. Use of DHEA and/or of its chemical and/or biological precursors and derivatives in a cosmetic composition for topical application to the skin, as agent for preventing the signs of skin ageing linked to atrophy of the epidermis.

4. Use according to any one of claims 1 to 3, characterized in that the said biological precursor is chosen from: Δ⁵-pregnenolone, 17α-hydroxypregnenolone and 17α-hydroxypregnenolone sulphate.

5. Use according to any one of claims 1 to 3, characterized in that the said biological derivative is chosen from: Δ⁵-androstene-3,17-diol and Δ⁴-androstene-3,17-dione.

6. Use according to any one of claims 1 to 3, characterized in that the said chemical derivative is chosen from: DHEA salts and DHEA esters.

7. Use according to claim 6, characterized in that the said DHEA salt is chosen from the water-soluble salts, such as DHEA sulphate.

8. Use according to claim 6, characterized in that the said DHEA ester is chosen from: esters of hydroxycarboxylic acids and of DHEA; DHEA salicylate; DHEA acetate; DHEA valerate; and DHEA enanthate.
